(19) United States
(12) Patent Application Publication
(10) Pub. No.: US 2014/0356231 A1
(43) Pub. Date: Dec. 4, 2014

(54) Automated KleenSan System

(71) Applicants: Emilia Rico-Munoz, Knoxville, TN (US); Shawn A. Johnson, Knoxville, TN (US)

(72) Inventors: Emilia Rico-Munoz, Knoxville, TN (US); Shawn A. Johnson, Knoxville, TN (US)

(21) Appl. No.: 14/293,801

(22) Filed: Jun. 2, 2014

Related U.S. Application Data
(60) Provisional application No. 61/829,837, filed on May 31, 2013.

(51) Int. Cl. A61L 2/18; A61L 2/02

(52) U.S. Cl. CPC A61L 2/18; A61L 2/02
USPC 422/37; 422/28; 422/292

(57) ABSTRACT

Systems and processes to dispense, clean and sanitize the key exposure points in the filling process to reduce risk of mold, yeast, or bacteria product spoilage, with minimal downtime. In some embodiments, a process uses a chemical solution that includes a liquid including potable water or another liquid—such as paramagnetic water, ultrapure water, R.O. water or any other kind of modified water—and a biocide solution such as sodium hypochlorite, stabilized chlorine dioxide, etc. at concentrations between 0.5 and 200 ppm or any of the components of electrochemical activated (EAC) water. In some embodiments, the water has a low enough chemical concentration to allow application without a follow-up rinse. The high ORP water at high pressure and flow removes the statically charged particles, preventing micro growth in between sprays, and effectively cleaning and sanitizing in a single step.

### CROSS-REFERENCE TO RELATED APPLICATIONS

This Application claims the benefit of U.S. Provisional Patent Application Ser. No. 61/829,837, filed May 31, 2013, the entirety of which is incorporated herein by reference.

### STATEMENT REGARDING FEDERALLY SPONSORED RESEARCH OR DEVELOPMENT

Not Applicable

### BACKGROUND OF THE INVENTION

1. Field of Invention

The present invention relates to systems and processes for cleaning and sanitizing equipment used in food preparation.

2. Description of the Related Art

Cleaning and sanitation of processing equipment fillers, conveyors, bottle rinsers, etc. take time, are costly and are, in many cases, ineffective. A reduction in sanitation time and increase in efficacy would generate water and sewage savings, chemical savings, personnel time cost reduction and increased production due to reduced down time.

### SUMMARY

The present invention includes, in some example embodiments, a process that involves a single-step or multi-step spraying of the key exposure points. The process uses a chemical solution that includes a liquid including potable water or another liquid—such as paramagnetic water, ultrapure water, R.O. water or any other kind of modified water—and a biocide solution such as sodium hypochlorite, stabilized chlorine dioxide, etc. at concentrations between 0.5 and 200 ppm or any of the components of electrochemical activated (EAC) water. Chemical concentration is measured using conductivity and ORP, a means of calculating concentration based on ORP and conductivity and a means stopping chemical concentration once the proper concentration is reached. In some embodiments, the water has a low enough chemical concentration to allow application without a follow-up rinse. The high ORP water at high pressure and flow removes the statically charged particles, preventing micro growth in between sprays, and effectively cleaning and sanitizing in a single step.

In some example embodiments of the present general inventive concept, an automated method of sterilizing food and beverage equipment includes applying to the equipment a chemical solution, said chemical solution including a liquid selected from the group consisting of potable water, paramagnetic water, ultrapure water, R.O. water, said chemical solution including a biocide solution, and applying to the equipment high ORP water at high pressure and flow, whereby statically charged particles are removed so as to prevent micro growth in between sprays, thereby effectively cleaning and sanitizing in a single step.

In some example embodiments the biocide solution includes sodium hypochlorite or stabilized chlorine dioxide.

In some example embodiments the biocide solution is present at concentrations between 0.5 and 200 ppm.

In some example embodiments the chemical solution and the ORP water are applied using spray bars, spray nozzles, rotating spray nozzles, or rotating spray balls.

In some example embodiments of the present general inventive concept, an automated system for sterilizing food and beverage equipment includes a tank to receive water and a sanitizer chemical, the water and the sanitizer chemical mixing in said tank to form chemical solution, said tank including an outlet; a centrifugal pump to receive chemical solution from said outlet of said tank; and a mixer to mix chemical solution from said centrifugal pump with cleaner solution.

In some example embodiments, said chemical solution including a biocide solution and includes a liquid selected from the group consisting of potable water, paramagnetic water, ultrapure water, and R.O. water.

In some example embodiments, said biocide solution includes sodium hypochlorite or stabilized chlorine dioxide.

In some example embodiments, said biocide solution is present at concentrations between 0.5 and 200 ppm

### BRIEF DESCRIPTION OF THE DRAWINGS

The above-mentioned and additional features of the invention will become more clearly understood from the following detailed description of the invention read together with the drawings in which:

FIG. 1 shows a block diagram illustrating one example embodiment of a cleaning skid according to the present general inventive concept.

### DETAILED DESCRIPTION

The present invention includes, in some example embodiments, a process that involves a single-step or multi-step spraying of the key exposure points. The process uses a chemical solution that includes a liquid including potable water or another liquid—such as paramagnetic water, ultrapure water, R.O. water or any other kind of modified water—and a biocide solution such as sodium hypochlorite, stabilized chlorine dioxide, etc. at concentrations between 0.5 and 200 ppm or any of the components of electrochemical activated (EAC) water. Chemical concentration is measured using conductivity and ORP, a means of calculating concentration based on ORP and conductivity and a means stopping chemical concentration once the proper concentration is reached. In some embodiments, the water has a low enough chemical concentration to allow application without a follow-up rinse. The high ORP water at high pressure and flow removes the statically charged particles, preventing micro growth in between sprays, and effectively cleaning and sanitizing in a single step.

In many of its several example embodiments, the present general inventive concept provides systems, methods, and processes to dispense, clean and sanitize the key exposure points in the filling process to reduce risk of mold, yeast, or bacteria product spoilage, with minimal downtime. Project savings are achieved by improvements in line efficiency, increase line time, reduction in outside support, reduction in chemical usage and reduction of customer complaints. The efficiency improvements arise from eliminating the practices, current in the known art, of stopping production once every 24-hour period to perform sanitation required in hot-fill processes. The present invention also provides a reduction in chemical usage achieved by a relatively short cleaning/sanitizing cycle compared to traditional methods. Additionally, a reduction in customer complaints and improved customer satisfaction are achieved due to significant reduction of microbiological activity measured on cleaned surfaces as compared to other cleaning methods.

The present invention is applicable to any process involving external cleaning of equipment: fillers, rinsers, cap chutes/tracks, conveyors, cooling rooms, cooling/warming tunnels, and so forth.

In some example embodiments, a process for sanitizing exterior equipment for beverage filling machines includes the following. The process includes a single-step or multi-step spraying of the key exposure points; e.g. external components of filler (valves, hoses, walls, bottle handling parts, etc.), rinser (grippers, transfer points) and cap handling equipment. The process includes the use of a chemical solution the includes a liquid including potable water, a liquid such as paramagnetic water, ultrapure water, R.O. water or any other kind of modified water, and a biocide solution such as sodium hypochlorite, stabilized chlorine dioxide, etc. at concentrations between 0.5 and 200 ppm or any of the components of electrochemical activated (EAC) water. Chemical concentration is measured using conductivity and ORP, a means of calculating concentration based on ORP and conductivity and a means stopping chemical concentration once the proper concentration is reached. The water used could have a low enough chemical concentration to allow application without a follow-up rinse. The high ORP water at high pressure and flow removes the sticky, sugary water, preventing micro growth in between sprays, and effectively cleaning and sanitizing in a single step. Cleaning is accomplished using installed spray bars, spray nozzles and rotating spray nozzles/spray balls.

In some example embodiments, a process for sanitizing a cap chute/track includes the following. The process includes a single-step/multi-step spraying of the key exposure points; e.g. guide rails, supports. The process uses a chemical solution that includes a liquid including potable water or another liquid—such as paramagnetic water, ultrapure water, R.O. water or any other kind of modified water—and a biocide solution such as sodium hypochlorite, stabilized chlorine dioxide, etc. at concentrations between 0.5 and 200 ppm or any of the components of electrochemical activated (EAC) water. Chemical concentration is measured using conductivity and ORP, a means of calculating concentration based on ORP and conductivity and a means stopping chemical concentration once the proper concentration is reached. In some embodiments, the water has a low enough chemical concentration to allow application without a follow-up rinse. The high ORP water at high pressure and flow removes the statically charged particles, preventing micro growth in between sprays, and effectively cleaning and sanitizing in a single step. Cleaning is accomplished using installed low volume misting spray nozzles (0.007 to 0.5 gpm).

In some example embodiments, a process for sanitizing rinsers includes the following. The process includes a single-step/multi-step spraying of the key exposure points; e.g. grippers, pans, support structures. The process uses a chemical solution that includes a liquid including potable water or another liquid—such as paramagnetic water, ultrapure water, R.O. water or any other kind of modified water—and a biocide solution such as sodium hypochlorite, stabilized chlorine dioxide, etc. at concentrations between 0.5 and 200 ppm or any of the components of electrochemical activated (EAC) water. Chemical concentration is measured using conductivity and ORP, a means of calculating concentration based on ORP and conductivity and a means stopping chemical concentration once the proper concentration is reached. In some embodiments, the water has a low enough chemical concentration to allow application without a follow-up rinse. The high ORP water at high pressure and flow removes the statically charged particles, preventing micro growth in between sprays, and effectively cleaning and sanitizing in a single step. Cleaning is accomplished using installed spray bars, spray nozzles and rotating spray nozzles/spray balls.

In some example embodiments, a process for sanitizing conveyors includes the following. The process includes a single-step/multi-step spraying of the key exposure points; e.g. guide rails, belts (stainless and plastic), supports and frames. The process uses a chemical solution that includes a liquid including potable water or another liquid—such as paramagnetic water, ultrapure water, R.O. water or any other kind of modified water—and a biocide solution such as sodium hypochlorite, stabilized chlorine dioxide, etc. at concentrations between 0.5 and 200 ppm or any of the components of electrochemical activated (EAC) water. Chemical concentration is measured using conductivity and ORP, a means of calculating concentration based on ORP and conductivity and a means stopping chemical concentration once the proper concentration is reached. In some embodiments, the water has a low enough chemical concentration to allow application without a follow-up rinse. The high ORP water at high pressure and flow removes the statically charged particles, preventing micro growth in between sprays, and effectively cleaning and sanitizing in a single step. Cleaning is accomplished using installed spray bars, spray nozzles and rotating spray nozzles/spray balls.

In some example embodiments, a process for sanitizing cooling/warming tunnels includes the following. The process includes a single-step/multi-step spraying of the key exposure points; e.g. belts, walls, ceilings and support structures. The process uses a chemical solution that includes a liquid including potable water or another liquid—such as paramagnetic water, ultrapure water, R.O. water or any other kind of modified water—and a biocide solution such as sodium hypochlorite, stabilized chlorine dioxide, etc. at concentrations between 0.5 and 200 ppm or any of the components of electrochemical activated (EAC) water. Chemical concentration is measured using conductivity and ORP, a means of calculating concentration based on ORP and conductivity and a means stopping chemical concentration once the proper concentration is reached. In some embodiments, the water has a low enough chemical concentration to allow application without a follow-up rinse. The high ORP water at high pressure and flow removes the statically charged particles, preventing micro growth in between sprays, and effectively cleaning and sanitizing in a single step. Cleaning is accomplished using installed spray bars, spray nozzles and rotating spray nozzles/spray balls.

In some embodiments, for beverage filling machines and other equipment including cap hoppers, bottle coolers, rinser, conveyors, floors, cap chutes/tracks that need in-depth cleaning and sanitizing, although the details of the installation will be different for each filling operation, the overall process and method includes the following:

A multi-step spraying of the key exposure points; e.g. external components of filler (valves, hoses, walls, bottle handling parts, etc.), rinser (grippers, transfer points), conveyors, cap handling equipment, etc.

Chemical solution consists of a liquid that will consist of potable water or any other liquid such as paramagnetic water, ultrapure water, R.O. water or any other kind of modified water and a cleaning solution such as sodium hydroxide, potassium hydroxide, alkaline products, acid products and surfactants at concentrations from 0.1% to 25.0%.

A rinse is conducted after the cleaning solution is sprayed and then a biocide solution such as sodium hypochlorite, stabilized chlorine dioxide, etc. at concentrations between 0.5 and 200 ppm or any of the components of electrochemical activated (EAC) water is sprayed.

Chemical concentration is measured using conductivity and ORP, a means of calculating concentration based on ORP and conductivity and a means stopping chemical concentration once the proper concentration is reached.

The water in some cases has a low enough chemical concentration to allow application without a follow-up rinse.

The high ORP water at high pressure and flow removes the sticky, sugary water, and food residues preventing micro growth in between sprays, and effectively cleaning and sanitizing in a single step.

Cleaning is accomplished using installed spray bars, spray nozzles and rotating spray nozzles/spray balls.

The present general inventive concept thus provides systems, methods, and processes to dispense, clean and sanitize the key exposure points in the filling process to reduce risk of mold, yeast, or bacteria product spoilage, with minimal downtime. Project savings are achieved by improvements in line efficiency, increase line time, reduction in outside support, reduction in chemical usage and reduction of customer complaints. A reduction in chemical usage is achieved by a relatively short cleaning/sanitizing cycle compared to prior methods known in the art. Additionally, a reduction in customer complaints and improved customer satisfaction are achieved due to significant reduction of microbiological activity measured on cleaned surfaces as compared to other cleaning methods.

In some example embodiments of the present general inventive concept, an automated cleaning system includes a skid system, which in turn includes a water/sanitizer tank, various pumps, PLC controller, measuring devices such as ORP meters, level sensors and conductivity meters, mixer/processor, and automatic valves. Sanitizer solutions are made up inside the water tank using a chemical metering pump at pre-determined concentrations. The water tank only has either water or sanitizer solutions. Cleaning solutions if needed are made up in-line after the water/sanitizer tank but before the mixer/processor. The chemical solutions are pumped from the tank using a centrifugal pump through a mixer/processor to the main line.

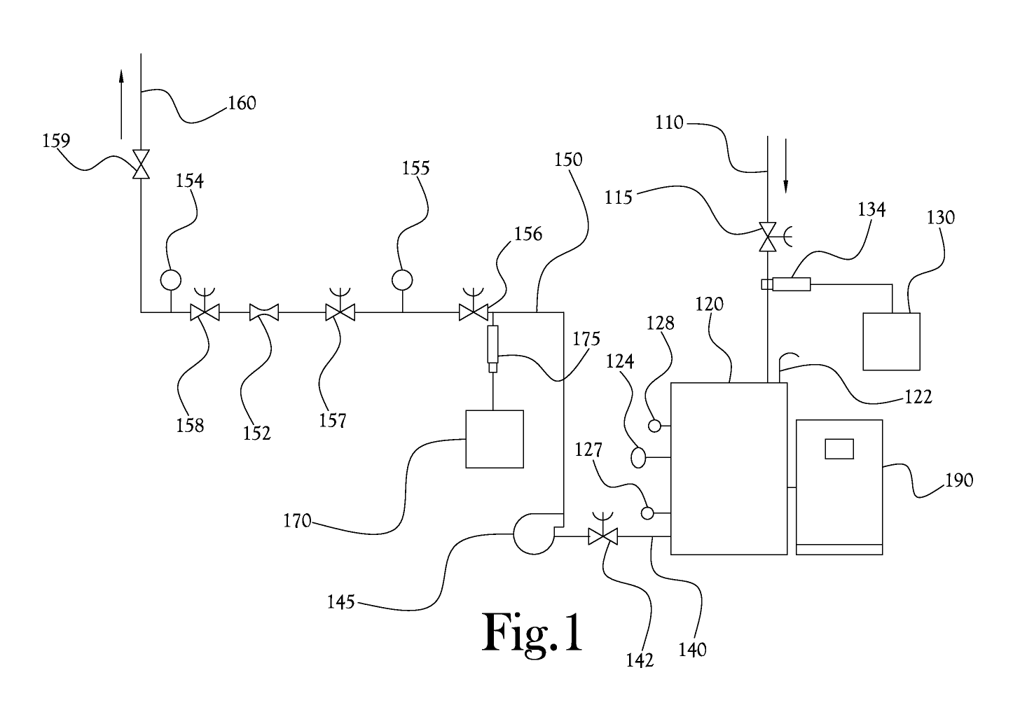

Turning to the Figure, FIG. 1 shows a block diagram illustrating one example embodiment of a cleaning skid according to the present general inventive concept. As shown in FIG. 1, the skid system includes a water inlet 110 (with valve 115) that feeds into a water/sanitizer tank 120 (which generally includes a vent 122). A sanitizer reservoir 130 supplies sanitizer chemical to the water/sanitizer tank 120, through a sanitizer chemical pump 134. In the illustrated example embodiment, an ORP meter 124 and level sensors 127, 128 are in an operative relationship with the water/sanitizer tank 120. The water/sanitizer tank 120 feeds through an outlet 140 (with safety valve 142) to a centrifugal pump 145, which directs chemical solutions and mixtures to the main line 150. The main line 150, which feeds to a system outlet 160, generally includes at least one processor or mixer 152, pressure gauges 154 and 155, and valves 156, 157, 158, 159. A low-foaming cleaner reservoir 170 also feeds into the main line 150 through a cleaner chemical pump 175. A PLC controller or control panel 190 permits overall control of the system. Sanitizer solutions are made up inside the water/sanitizer tank 120 using a chemical metering pump at pre-determined concentrations. The water/sanitizer tank 120 only has either water or sanitizer solutions. Cleaning solutions if needed are made up in-line after the water/sanitizer tank 120 but before the mixer/processor 152.

In some example embodiments of the present general inventive concept, an automated cleaning system includes a cap chute/track, generally including a single-step/multi-step spraying of the key exposure points (e.g. guide rails, supports); chemical solution including a liquid that will includes potable water or any other liquid such as paramagnetic water, ultrapure water, R.O. water or any other kind of modified water and a biocide solution such as sodium hypochlorite, stabilized chlorine dioxide, etc. at concentrations between 0.5 and 100 ppm or any of the components of electrochemical activated (EAC) water. Chemical concentration is measured using conductivity and ORP, a means of calculating concentration based on ORP and conductivity and a means stopping chemical concentration once the proper concentration is reached. In some embodiments, the water has a low enough chemical concentration to allow application without a follow-up rinse. High ORP water at high pressure and flow removes statically charged particles, preventing micro growth in between sprays, and effectively cleaning and sanitizing in a single step. A catch pan catches overflow and prevents over spray on personnel. Cleaning is accomplished using installed low volume misting spray nozzles (0.007 to 0.5 gpm).

In some example embodiments of the present general inventive concept, an automated cleaning system includes a cap elevator conveyor system, which generally includes a single-step/multi-step spraying of the key exposure points (e.g., guide rails, belts [stainless and plastic], supports and frames); chemical solution including of a liquid that includes potable water or any other liquid such as paramagnetic water, ultrapure water, R.O. water or any other kind of modified water and a biocide solution such as sodium hypochlorite, stabilized chlorine dioxide, etc. at concentrations between 0.5 and 100 ppm or any of the components of electrochemical activated (EAC) water. Chemical concentration is measured using conductivity and ORP, a means of calculating concentration based on ORP and conductivity and a means stopping chemical concentration once the proper concentration is reached. In some embodiments, the water has a low enough chemical concentration to allow application without a follow-up rinse. High ORP water at high pressure and flow removes the statically charged particles, preventing micro growth in between sprays, and effectively cleaning and sanitizing in a single step. Spray bars with misting spray nozzles are placed at the top of the cap elevator to spray the elevator as the elevator moves past. Cleaning is accomplished using installed spray bars, spray nozzles and rotating spray nozzles/spray balls.

In some example embodiments of the present general inventive concept, an automated cleaning system includes a cap hopper conveyor system, which generally includes a single-step/multi-step spraying of the key exposure points (e.g., guide rails, belts [stainless and plastic], supports and frames); chemical solution including of a liquid that includes potable water or any other liquid such as paramagnetic water, ultrapure water, R.O. water or any other kind of modified water and a biocide solution such as sodium hypochlorite, stabilized chlorine dioxide, etc. at concentrations between 0.5 and 100 ppm or any of the components of electrochemical activated (EAC) water. Chemical concentration is measured using conductivity and ORP, a means of calculating concentration based on ORP and conductivity and a means stopping chemical concentration once the proper concentration is reached. In some embodiments, the water has a low enough chemical concentration to allow application without a follow-up rinse. High ORP water at high pressure and flow removes the statically charged particles, preventing micro growth in between sprays, and effectively cleaning and sanitizing in a single step. Spray bars with misting spray nozzles are placed at the top of the cap elevator to spray the elevator as the elevator moves past. Cleaning is accomplished using installed spray bars, spray nozzles and rotating spray nozzles/spray balls.

In some example embodiments of the present general inventive concept, an automated cleaning system includes a conveyor system, which generally includes a single-step/multi-step spraying of the key exposure points (e.g., guide rails, belts [stainless and plastic], supports and frames); chemical solution including of a liquid that includes potable water or any other liquid such as paramagnetic water, ultrapure water, R.O. water or any other kind of modified water and a biocide solution such as sodium hypochlorite, stabilized chlorine dioxide, etc. at concentrations between 0.5 and 100 ppm or any of the components of electrochemical activated (EAC) water. Chemical concentration is measured using conductivity and ORP, a means of calculating concentration based on ORP and conductivity and a means stopping chemical concentration once the proper concentration is reached. In some embodiments, the water has a low enough chemical concentration to allow application without a follow-up rinse. High ORP water at high pressure and flow removes the statically charged particles, preventing micro growth in between sprays, and effectively cleaning and sanitizing in a single step. Spray bars with misting spray nozzles are placed at the top of the cap elevator to spray the elevator as the elevator moves past. Cleaning is accomplished using installed spray bars, spray nozzles and rotating spray nozzles/spray balls.

In some example embodiments of the present general inventive concept, an automated cleaning system includes an egg roll wrapping conveyor system, generally including a single-step/multi-step spraying of the key exposure points (e.g., guide rails, belts [stainless and plastic], supports and frames); chemical solution including a liquid that includes potable water or any other liquid such as paramagnetic water, ultrapure water, R.O. water or any other kind of modified water and a biocide solution such as sodium hypochlorite, stabilized chlorine dioxide, etc. at concentrations between 0.5 and 100 ppm or any of the components of electrochemical activated (EAC) water. Chemical concentration is measured using conductivity and ORP, a means of calculating concentration based on ORP and conductivity and a means stopping chemical concentration once the proper concentration is reached. In some embodiments, this water has a low enough chemical concentration to allow application without a follow-up rinse. High ORP water at high pressure and flow removes the statically charged particles, preventing micro growth in between sprays, and effectively cleaning and sanitizing in a single step. A stainless steel enclosure with mounted rotating spray balls that the egg roll wrapping components pass through to be cleaned and/or sanitized. The enclosure allows the wrapping components to be sanitized/rinsed while the equipment is running A second stainless steel enclosure with air blows is installed after the first to facilitate drying. Cleaning is accomplished using installed spray bars, spray nozzles and rotating spray nozzles/spray balls.

In some example embodiments of the present general inventive concept, an automated cleaning system includes a filler system, generally including a single-step/multi-step spraying of the key exposure points; e.g. external components of filler (valves, hoses, walls, bottle handling parts, etc.), rinser (grippers, transfer points) and cap handling equipment; along with a chemical solution including a liquid that includes potable water or any other liquid such as paramagnetic water, ultrapure water, R.O. water or any other kind of modified water and a biocide solution such as sodium hypochlorite, stabilized chlorine dioxide, etc. at concentrations between 0.5 and 100 ppm or any of the components of electrochemical activated (EAC) water. Chemical concentration is measured using conductivity and ORP, a means of calculating concentration based on ORP and conductivity and a means stopping chemical concentration once the proper concentration is reached. In some embodiments, the water could have a low enough chemical concentration to allow application without a follow-up rinse. High ORP water at high pressure and flow removes the sticky, sugary water, preventing micro growth in between sprays, and effectively cleaning and sanitizing in a single step. Cleaning is accomplished using installed spray bars, spray nozzles and rotating spray nozzles/spray balls. An automatic dump valve on the piping is installed to speed up drainage time at the end of the cleaning cycle so spray balls and nozzles do not drip into product.

In some example embodiments of the present general inventive concept, an automated cleaning system includes a rinser system, generally including a single-step/multi-step spraying of the key exposure points; e.g. grippers, pans, support structures; together with a chemical solution including a liquid that includes potable water or any other liquid such as paramagnetic water, ultrapure water, R.O. water or any other kind of modified water and a biocide solution such as sodium hypochlorite, stabilized chlorine dioxide, etc. at concentrations between 0.5 and 100 ppm or any of the components of electrochemical activated (EAC) water. Chemical concentration is measured using conductivity and ORP, a means of calculating concentration based on ORP and conductivity and a means stopping chemical concentration once the proper concentration is reached. In some embodiments, this water has a low enough chemical concentration to allow application without a follow-up rinse. High ORP water at high pressure and flow removes the statically charged particles, preventing micro growth in between sprays, and effectively cleaning and sanitizing in a single step. Cleaning is accomplished using installed spray bars, spray nozzles and rotating spray nozzles/spray balls.

In some example embodiments of the present general inventive concept, an automated cleaning system includes a spiral cooling conveyor system, generally including a single-step/multi-step spraying of the key exposure points; e.g. guide rails, belts (stainless and plastic), supports and frames; together with a chemical solution including a liquid that includes potable water or any other liquid such as paramagnetic water, ultrapure water, R.O. water or any other kind of modified water and a biocide solution such as sodium hypochlorite, stabilized chlorine dioxide, etc. at concentrations between 0.5 and 100 ppm or any of the components of electrochemical activated (EAC) water. Chemical concentration is measured using conductivity and ORP, a means of calculating concentration based on ORP and conductivity and a means stopping chemical concentration once the proper concentration is reached. In some embodiments, this water has a low enough chemical concentration to allow application without a follow-up rinse. High ORP water at high pressure and flow removes the statically charged particles, preventing micro growth in between sprays, and effectively cleaning and sanitizing in a single step. Spray balls are positioned around the spiral cooling conveyor in vertical positions around the conveyor. One or more stainless steel enclosures are installed if needed to prevent overspray protecting personnel and other equipment. Cleaning is accomplished using installed spray bars, spray nozzles and rotating spray nozzles/spray balls.

In some example embodiments of the present general inventive concept, an automated cleaning system includes a tortilla cooling conveyor system, generally including a single-step/multi-step spraying of the key exposure points; e.g. guide rails, belts (stainless and plastic), supports and frames; together with a chemical solution including a liquid that includes potable water or any other liquid such as paramagnetic water, ultrapure water, R.O. water or any other kind of modified water and a biocide solution such as sodium hypochlorite, stabilized chlorine dioxide, etc. at concentrations between 0.5 and 100 ppm or any of the components of electrochemical activated (EAC) water. Chemical concentration is measured using conductivity and ORP, a means of calculating concentration based on ORP and conductivity and a means stopping chemical concentration once the proper concentration is reached. In some embodiments, this water has a low enough chemical concentration to allow application without a follow-up rinse. High ORP water at high pressure and flow removes the statically charged particles, preventing micro growth in between sprays, and effectively cleaning and sanitizing in a single step. Spray bars with spray nozzles are placed between cooling conveyor belts so that complete and overlapping coverage is obtained. Spray bars with spray nozzles blowing air are placed between cooling conveyor belts to facilitate drying. Cleaning is accomplished using installed spray bars, spray nozzles and rotating spray nozzles/spray balls. Rotating spray balls are placed in the ceiling to clean/sanitize walls, refrigeration units, floors, ceiling and equipment frames.

While the present invention has been illustrated by description of several embodiments and while the illustrative embodiments have been described in considerable detail, it is not the intention of the applicant to restrict or in any way limit the scope of the appended claims to such detail. Additional advantages and modifications will readily appear to those skilled in the art. The invention in its broader aspects is therefore not limited to the specific details, representative apparatus and methods, and illustrative examples shown and described. Accordingly, departures may be made from such details without departing from the spirit or scope of applicant's general inventive concept.

What is claimed is:

1. An automated method of sterilizing food and beverage equipment comprising:
applying to the equipment a chemical solution, said chemical solution including a liquid selected from the group consisting of potable water, paramagnetic water, ultrapure water, R.O. water, said chemical solution including a biocide solution; and
applying to the equipment high ORP water at high pressure and flow, whereby statically charged particles are removed so as to prevent micro growth in between sprays, thereby effectively cleaning and sanitizing in a single step.

2. The automated method of claim 1 wherein said biocide solution includes sodium hypochlorite or stabilized chlorine dioxide.

3. The automated method of claim 1 wherein said biocide solution is present at concentrations between 0.5 and 200 ppm.

4. The automated method of claim 1 wherein said chemical solution and said ORP water are applied using spray bars, spray nozzles, rotating spray nozzles, or rotating spray balls.

5. An automated system for sterilizing food and beverage equipment comprising:
a tank to receive water and a sanitizer chemical, the water and the sanitizer chemical mixing in said tank to form chemical solution, said tank including an outlet;
a centrifugal pump to receive chemical solution from said outlet of said tank; and
a mixer to mix chemical solution from said centrifugal pump with cleaner solution.

6. The automated system of claim 5 wherein said chemical solution including a biocide solution and includes a liquid selected from the group consisting of potable water, paramagnetic water, ultrapure water, and R.O. water.

7. The automated system of claim 6 wherein said biocide solution includes sodium hypochlorite or stabilized chlorine dioxide.

8. The automated system of claim 6 wherein said biocide solution is present at concentrations between 0.5 and 200 ppm
